(19) United States
(12) Patent Application Publication
(10) Pub. No.: US 2007/0010583 A1
(43) Pub. Date: Jan. 11, 2007

(54) Methods and compositions for reducing blood homocysteine levels

(75) Inventors: Julia Dibner, Chesterfield, MO (US); Charles S. Schasteen, St. Louis, MO (US); Mercedes Vazquez-Anon, Chesterfield, MO (US)

Correspondence Address: PALMER & DODGE, LLP;KATHLEEN M. WILLIAMS, 111 HUNTINGTON AVENUE, BOSTON, MA, 02199, US

(73) Assignee: Novus International, Inc.

(21) Appl. No.: 11/441,490

(22) Filed: May 25, 2006

Related U.S. Application Data
(60) Provisional application No. 60/684,549, filed on May 25, 2005.

(51) Int. Cl. A61K 31/19

(52) U.S. Cl. 514/557

(57) ABSTRACT

The invention provides methods for reducing blood homocysteine levels in mammals, and treating or preventing diseases associated with elevated blood homocysteine levels, such as cardiovascular diseases and cognitive disorders. The invention also provides nutritional and pharmaceutical compositions comprising 2-hydroxy-4-(thiomethyl)-butanoic acid, including esters, analogs, derivatives or complex thereof.

### RELATED APPLICATIONS

This application claims the benefit of U.S. provisional application 60/684,549, filed May 25, 2005. The aforementioned application is hereby incorporated by reference in its entirety.

### FIELD OF THE INVENTION

This invention relates to methods and compositions for reducing blood homocysteine levels in mammals, and for treating diseases and disorders associated with elevated homocysteine levels.

### BACKGROUND OF THE INVENTION

Numerous diseases are correlated with elevated serum homocysteine (“Hcy”) levels. For example, a moderate elevation in plasma total homocysteine (“tHcy”) levels is strongly and independently correlated with an increased risk of numerous vascular diseases (Hankey and Eikelboom, Lancet (1999) 354:407-413). In addition, a growing body of research suggests a link between elevated levels of homocysteine and an increased risk of cognitive dysfunction, including development of Alzheimer's disease (Seshadri et al., (2002) NEJM 346: 476-483). Homocysteine, which is an intermediate in the metabolism of methionine, an essential amino acid, can be metabolized by two distinct pathways: a re-methylation pathway to regenerate methionine, and a trans-sulfuration pathway, which degrades homocysteine into cysteine, and eventually into taurine. Multiple factors contribute to elevated homocysteine levels (“hyperhomocysteinemia”), including genetics and diet. For example, elevated homocysteine levels can be caused by a deficiency of folic acid, vitamin B12, and/or vitamin B6, all of which are co-factors required during the metabolism of methionine. A transient increase in homocysteine levels is also observed shortly after a methionine-rich diet (“methionine load”). Given the well-known medical risks associated with elevated blood levels of homocysteine, normalization of serum homocysteine levels represents an important medical goal, both from a therapeutic and prophylactic perspective.

### SUMMARY OF THE INVENTION

The present invention is based in part on the surprising discovery that administration of HMTBA, for example as a replacement for dietary methionine, can lower the blood homocysteine levels in mammals. Therefore, disclosed herein are methods and compositions comprising HMTBA for reducing blood homocysteine levels, treating diseases associated with elevated blood homocysteine levels, and methods and compositions for simultaneously reducing blood homocysteine levels and supplying an essential amino acid and mineral to a mammal.

In one aspect, the invention provides a method of treating a disease or disorder associated with elevated homocysteine levels in a subject. The method comprises administering HMTBA, its salts, chelates or complexes thereof, in a dose sufficient to lower homocysteine levels in the blood.

In another aspect, the invention provides a method of administering a mineral to a human, comprising administering a mineral chelate composition comprising 2-hydroxy-4-(thiomethyl)-butanoic acid.

In still another aspect, the invention provides a method of preventing and/or treating a condition associated with mineral deficiency in a human or other animal subject comprising orally administering an effective amount of a mineral comprising a salt or complex selected from the group consisting of calcium-2-hydroxy-4-(thiomethyl)-butanoic acid, zinc-2-hydroxy-4-(thiomethyl)-butanoic acid, magnesium-2-hydroxy-4-(thiomethyl)-butanoic acid, selenium-2-hydroxy-4-(thiomethyl)-butanoic acid, and iron-2-hydroxy-4-(thiomethyl)-butanoic acid. In another aspect, the invention provides a composition useful for the treatment of diseases and disorders associated with elevated homocysteine levels. The composition comprises HMTBA, its salts, chelates or complexes thereof, in a dose sufficient to lower blood homocysteine levels. Such a composition can be in the form of a pharmaceutical or nutritional composition, such as a nutritional supplement (e.g., in powder, tablet or liquid form), a sports bar or candy, beverage, or a food additive.

### DETAILED DESCRIPTION OF THE INVENTION

I. Definitions

As used herein, “homocysteine” and “Hcy” refer to a compound with the empirical formula: C₄H₉NO₂S and molecular weight of 135.18, and CAS Number 454-28-4. Biologically, homocysteine is produced by demethylation of methionine and is an intermediate in the biosynthesis of cysteine from methionine. The term “homocysteine” encompasses free homocysteine (in the reduced form) and conjugated homocysteine (in the oxidized form). Homocysteine can conjugate with proteins, peptides, itself or other thiols through a disulfide bond.

As used herein, an “elevated homocysteine level” refers to a concentration of homocysteine in the blood or a blood fraction (e.g., serum or plasma) of a mammal (human or animal) that is increased relative to the normal or average concentration of homocysteine for that mammal. For example, and depending on the context, an “elevated homocysteine level” refers to a concentration of homocysteine in the serum or plasma of a subject that is (1) higher than the concentration of homocysteine in the blood or blood fraction of an average subject (i.e., a hypothetical subject having the average concentration of homocysteine for individuals of the same species, gender and age); (2) higher than the blood homocysteine level in the upper tertile for control subjects of the same species, gender and age; and/or (3) higher than the average homocysteine blood levels of normal or control subjects of the same species, gender and age. An “elevated homocysteine level” will typically be at least at least 3%, 5%, 10%, 15%, 20%, 25%, 50%, or 60% or more above the level of homocysteine in the serum or plasma of an average or control subject. In one embodiment, an “elevated homocysteine level” means at least 15% above the average or control level of homocysteine in the serum or plasma. In the context of the present invention, a “moderate elevation in homocysteine level” means 15 to 30 μmol/L above the average or control level of the homocysteine in the serum or plasma, “an intermediate elevation in homocysteine level” means 30 to 100 μmol/L above the average or control level of the homocysteine in the serum or plasma, and a “severe elevation in homocysteine level” means >100 μmol/L above the average or control level of the homocysteine in the serum or plasma, as measured during fasting.

As used herein, “reducing a subject's total methionine intake” means reducing the total daily amount of methionine (both free and dietary) ingested by the subject, including methionine incorporated within protein. The reduction in a subject's total methionine intake can be calculated on a percentage basis, for example, a reduction in total daily intake of at least 15%, 20%, 25%, 30%, 40%, 50%, 60%, 70%, 80%, 90%, 95%, 99% or 100%. Alternatively, the reduction in a subject's total methionine intake can be measured by weight, for example, at least 2,000 mg, 1,400 mg, 1,000 mg, 800 mg, 500 mg, 300 mg, 200 mg, 100 mg, 75 mg, 50 mg, 25 mg, or 10 mg or more per day.

As used herein, the terms “HMTBA” and “2-hydroxy-4-(methylthio)butanoic acid” are used interchangeably to refer to 2-hydroxy-4-(methylthio)butanoic acid, including any ester, analog, or derivative thereof. As used herein, the terms “2-hydroxy-4-(thiomethyl)-butanoic acid complex,” “HMTBA complex” and “HMTBA-containing complex” refer to a complex containing 2-hydroxy-4-(thiomethyl)-butanoic acid, including, without limitation, a complex comprising HMTBA and a cation, salt, chelate, or solvate, and mixtures or combinations thereof. In certain contexts, the complexes refer to 2-hydroxy-4-(thiomethyl)-butanoic acid. As used herein, the term “mineral chelate of HMTBA” refers to a complex comprising HMTBA and a mineral, such as an essential mineral. HMTBA and HMTBA complexes useful for the practice of the present invention are available in various forms, as discussed in detail below.

As used herein, the terms an “essential amino acid” and “essential mineral” refer to any amino acid or mineral, respectively, that is required for life and/or growth of the mammal. Essential amino acids for humans include leucine, isoleucine, valine, methionine, threonine, lysine, histidine, phenylalanine, and tryptophan.

As used herein, a “disease associated with elevated homocysteine levels” refers to a disorder or disease whose onset and/or progression is associated with an elevated blood (e.g., serum or plasma) homocysteine level, and includes, without limitation, hyperhomocysteinemia, hyperhomocysteineuria, occlusive vascular diseases such as myocardial infarction, stroke, pulmonary embolism, occlusive disease of cerebral, carotid and aorto-iliac vessels, Alzheimer's disease, and neurological degeneration. Several of these diseases are discussed in further detail below.

As used herein, a “condition associated with mineral deficiency” in a human refers to a disorder or disease whose cause and/or progression is associated with deficiency of a mineral, for example calcium, magnesium, zinc, manganese, iron, selenium, or copper. As used herein, a “condition associated with mineral deficiency” in a human includes, without limitation, osteoporosis, hypertension, bone loss, elevated serum or plasma levels of low density lipoprotein, dilated cardiomyopathy, hypocalcemia, hypokalemia, Menke's disease, anemia, hyperthyroidism, hemorrhoids, anorexia, growth retardation, delayed sexual maturation, hypogonadism and hypospermia, alopecia, immune disorders, dermatitis, night blindness, impaired taste (hypogeusia), impaired wound healing, acrodermatitis enteropathica, and Keshan disease.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, when used in the context of modulating homocysteine levels, an “effective amount” refers to that amount of a modulator of serum homocysteine levels (e.g., HMTBA) effective to produce the intended pharmacological, therapeutic or preventive result. For example, if a given clinical treatment is considered effective when there is at least a 25% reduction in a disease symptom, a therapeutically effective amount is the amount necessary to effect at least a 25% reduction in that parameter. The exact amount required will vary from subject to subject, depending on the species, age, general condition of the subject, the particular delivery form, bioavailability, and the like.

As used herein, an “effective amount” of a mineral refers to a quantity which is sufficient to yield a desired therapeutic response. The specific “effective amount” will vary depending on factors such as the particular condition being treated, the physical condition of the patient, the length of treatment, and the specific formulations employed.

As used herein, a “safe and effective amount” of a mineral refers to a quantity which is sufficient to yield a desired therapeutic response without undue adverse side effects (such as toxicity, irritation, or allergic responses), commensurate with a reasonable risk/benefit ratio when used in the manner described herein. The specific “safe and effective amount” will vary depending on factors such as the particular condition being treated, the physical condition of the patient, the length of treatment, and the specific formulations employed.

II. HMTBA

Various forms of 2-hydroxy-4-methylthiobutanoic acids are known and readily available in the art. Such forms include the individual D- and L-isomers of 2-hydroxy-4-methylthiobutanoic acid, as well as racemic mixtures thereof. A D-isomer of 2-hydroxy-4-methylthiobutanoic acid can be readily converted to the L-isomer, and vice versa, by procedures known in the art. Hence, for purposes of the present invention, the terms “HMTBA” and “2-hydroxy-4-methylthiobutanoic acid” refer to either the D- or L-isomer of 2-hydroxy-4-methylthiobutanoic acid or any mixture thereof. The usual commercial form of 2-hydroxy-4-methylthiobutanoic acid (HMTBA) is the optically racemic D,L-2-hydroxy-4-methylthiobutanoic acid mixture. Liquid forms of 2-hydroxy-4-methylthiobutanoic acid (typically 88% minimum by weight) are available from several commercial sources, including Novus International, St. Louis, Mo. (ALIMET™) and Adisseo, Antony, France (RHODIMET™ AT 88).

In the context of the present invention, HMTBA includes esters of 2-hydroxy-4-(methylthio)butanoic acid, and complexes comprising esters of 2-hydroxy-4-(methylthio)butanoic acid. Representative esters of HMTBA include, for example, methyl, ethyl, n-propyl, isopropyl, butyl esters (including n-butyl, sec-butyl, isobutyl, and tert-butyl), pentyl esters (including n-pentyl and isopentyl), and hexyl esters (including n-hexyl and isohexyl). The esters can be linear and branched chain alkyl esters. The present invention further contemplates the use of diesters of 2-hydroxy-4-(methylthio)butanoic acid, as well as complexes comprising such diesters. Esters of 2-hydroxy-4-(methylthio)butanoic acid can be produced by methods known in the art, for example, the methods described in U.S. 2004/0154549, U.S. Pat. No. 4,782,173, and U.S. Pat. No. 4,388,327, which are incorporated by reference in their entireties herein. Esters of 2-hydroxy-4-(methylthio)butanoic acid can have the advantage that its absorption is more rapid and/or efficient, compared with unesterified 2-hydroxy-4-(methylthio)butanoic acid, thereby increasing its overall bioavailability.

The present invention also contemplates the use of HMTBA complexes and salts. In one embodiment, the HMTBA is a metal complex or mineral chelate of HMTBA. Examples of mineral useful for the practice of the present invention include, without limitation, sodium, potassium, calcium, cobalt, copper, iron, magnesium, manganese, selenium, chromium, and zinc. calcium-HMTBA complex and zinc-HMTBA complexes. Methods for preparing HMTBA complexes and salts are known in the art, and are described, for example, in U.S. Pat. No. 2,745,745, U.S. Pat. No. 2,938,053, U.S. Pat. No. 4,579,962, U.S. Pat. No. 4,335,257, U.S. Pat. No. 6,461,664,), U.S. Pat. No. 5,583,243 and U.S. Ser. No. 11/074,387, the contents of which are incorporated by reference in their entireties herein.

III. Compositions Comprising HMBTA

In one aspect, the present invention provides compositions for reducing the blood levels of homocysteine in a mammal. Compositions useful for reducing blood homocysteine levels comprise HMTBA and HMTBA complexes, as described above. Such compositions may be in the form of a nutritional composition, such as a nutritional supplement (e.g., in powder, tablet or liquid form), a sports bar or candy, beverage, or a food additive. As discussed above, HMTBA is an analog of methionine, an essential amino acid. HMTBA has been shown to be as effective as methionine, an essential amino acid, for nutritional purposes in animals. HMTBA and HMTBA complexes have favorable absorption profiles, and rapidly convert into L-methionine upon absorbed into the tissue of animals. HMTBA is also an effective carrier for metals. As a result, in addition to providing a source of methionine, HMTBA can be used as a chelate or a carrier for essential minerals. Mineral chelates of HMTBA show excellent bioavailability.

Supplementation of HMTBA in animals has been shown to result in optimal growth and muscle accretion. Since HMTBA provides a source of the essential amino acid methionine, and, as taught herein, reduces blood homocysteine levels when used as a replacement for dietary methionine, the HMTBA compositions of the present invention provide an excellent nutritional supplement for athletes, body builders and others. Therefore, in one aspect, the present invention provides nutritional supplement compositions comprising HMTBA. The HMTBA can be 2-hydroxy-4-(methylthio)butanoic acid, or a HMTBA-containing complex, as described above. The HMTBA-containing complex can be a mineral chelate comprising a metallic cation and HMTBA. In one embodiment, the composition further comprises amino acids. The amino acids can be any amino acid, and are preferably selected from the group consisting of arginine, asparagine, aspartic acid, alanine, glutamic acid, glutamine, glycine, leucine, isoleucine, methionine, valine, threonine, lysine, histidine, phenylalanine, and tryptophan.

The composition can additionally comprise a phospholipid, a carbohydrate, and protein material. The protein material can be derived from wheat proteins, rice proteins, pea proteins, casein proteins, whey proteins or mixtures thereof. In one embodiment, the protein material contained within a single serving size of a composition is substantially devoid of methionine. For example, the methionine content in a serving size may be 350 mg or less, 300 mg or less, 250 mg or less, 200 mg or less, 150 mg or less, 100 mg or less, 50 mg or less, or 10 mg or less.

In another embodiment, the composition is in the form of a chewable or non-chewable tablet, an energy bar, a sports bar, a weight loss bar, a snack bar, a granola bar, an isotonic beverage, a drink mix, an energy drink, a sports drink, a citrus drink, a fruit drink, a carbonated drink, or the like.

In yet another embodiment, the invention provides a mineral-supplemented fluid composition comprising a HMTBA-containing mineral complex, such as an isotonic beverage. The HMTBA-containing complex can be dissolved in an ingestive liquid, wherein the mineral-supplemented fluid composition has about 10% to about 200% of the recommended daily allowance (RDA) of the mineral per serving. The mineral can be selected from the group consisting of potassium, calcium, cobalt, copper, iron, magnesium, manganese, and zinc.

In still another embodiment, the composition comprises a combination of HMTBA and other active agents to further facilitate the lowering of homocysteine levels, such as co-factors required for the metabolism of homocysteine. Such active agents include, for example, vitamin B12 and folic acid, which have been shown to affect blood homocysteine levels. Thus, in one embodiment, the composition comprising HMTBA and a vitamin or co-factor selected from the group consisting of vitamin B6, folic Acid, vitamin B12, and inositol, betaine, trimethylglycin, cyanobalamin, cystine, serine, copper, and other B-group vitamins. While the optimum concentration of vitamin or co-factor will vary depending on the diet, health, and age of the recipient, and other environmental or genetic factors, typical concentrations of the vitamins and co-factors are as follows: Vitamin B6 (pyroxidine HCl and Pyroxidal 5-Phosphate): between 5 and 5,000 mg, for example between 10 mg and 2000 mg, 40 mg and 1000 mg, or 100 mg and 500 mg; Folic acid: 200 μg and 40 mg, for example between 500 μg and 20 mg, or 1 mg and 10 mg; Vitamin B12: between 10 μg and 40 mg, for example between 25 μg and 20 mg, 50 μg and 10 mg, or 100 μg and 5 mg; and Inositol: between 10 to 5,000 mg, for example between 25 mg and 2,000 mg, between 50 mg and 1,000 mg, or 100 mg and 400 mg.

The compositions of the present invention can also include any multi-vitamin and mineral supplement for use in lowering homocysteine. Examples of multivitamin and mineral supplements that can be used in the compositions of the present invention, include, but are not limited to, those described in U.S. Pat. No. 6,361,800; U.S. Pat. No. 6,353,003; U.S. Pat. No. 6,323,188; U.S. Pat. No. 6,274,170; U.S. Pat. No. 6,210,686; U.S. Pat. No. 6,203,818; and U.S. Pat. No. 5,668,173, each of which is incorporated by reference in its entirety herein.

In another embodiment, the compositions further comprise cystine and/or serine. Cystine concentrations will generally be between 150 mg and 3,000 mg, between 200 mg and 2,000 mg, or between 400 mg and 1,300 mg. Likewise, serine concentrations will generally range between 500 mg and 16,000 mg, between 1,000 mg and 8,000 mg, or between 2,000 mg and 4,000 mg.

IV. Methods for Reducing Blood Homocysteine Levels

In one aspect, the present invention provides methods for reducing the blood levels of homocysteine in a mammal. The methods comprise administering an amount of HMTBA or an HMTBA complex, including compositions comprising HMTBA and/or HMTBA complexes, sufficient to lower the concentration of homocysteine in the blood of the mammal. For example, the HMTBA can be administered at a dosage of 200 mg, 300 mg, 400 mg, 500 mg, 600 mg, 750 mg, 1,000 mg, 1,250 mg, 1,500 mg, 2,000 mg, 2,500 mg, 3,000 mg, 3,500 mg, 4,000 mg, 5,000 mg, or 7,000 mg or more per day.

In another aspect, the present invention provides methods for reducing elevated levels of homocysteine, as well as the adverse effects of elevated levels of homocysteine, in a mammal. An elevated homocysteine level, known as hyperhomocysteinemia, is typically defined as a serum homocysteine level of about 15 μmol/L, and may be caused by genetic defects, renal insufficiency, certain drugs, or nutritional deficiencies of folate, vitamin B₆, or vitamin B₁₂. As described elsewhere herein, a moderate elevation in serum homocysteine has been found to be strongly and independently associated with numerous diseases, including vascular diseases and a reduction in cognitive function. As discussed above, serum homocysteine levels increase upon dietary supplementation of methionine, an essential amino acid. Homocysteine is an intermediate in the synthesis of cysteine from methionine.

In one embodiment, the HMTBA is administered to a subject such that HMTBA partially or totally replaces the methionine intake by the subject. Methionine is a precursor to homocysteine synthesis. Homocysteine levels are known to elevate immediately following periods of methionine load (REF). Surprisingly, the present inventors have discovered that periods of HMTBA load, unlike periods of methionine load, result in a decrease in the homocysteine peaks. In general, the higher the HMTBA intake relative to the methionine intake, the lower the level of homocysteine in the blood. The HMTBA to methionine ratio in the subject's diet may be adjusted as appropriate for maximum nutritional and/or medical benefits, depending on the subject's age, general health, and nutritional and medical requirements. For example, the HMTBA may be administered to a subject at a ratio of at least 10%, 15%, 20%, 25%, 30%, 40%, 50%, 60%, 70%, 80%, 90%, 95%, 99% or 100% of the total methionine intake, measured on a molar basis, thereby reducing the subject's total methionine intake by at least 10%, 15%, 20%, 25%, 30%, 40%, 50%, 60%, 70%, 80%, 90%, 95%, 99% and 100%, respectively. In one embodiment, the total HMTBA intake is at 10% of the total methionine intake. In another embodiment, the total HMTBA intake is at 20% of the total methionine intake. Higher HMTBA intake is typically indicated for treating diseases and disorders associated with elevated homocysteine levels, as discussed below.

In another embodiment, the method comprises reducing a subject's intake of methionine to a defined maximum amount per day. According to this embodiment, the subject's intake of methionine is limited to 2,000 mg, 1,800 mg, 1,600 mg, 1,400 mg, 1,200 mg, 1,000 mg, 800 mg, 600 mg, 500 mg, 400 mg, 300 mg, 200 mg, or 100 mg or less per day. In another embodiment, the method comprises reducing the subject's intake of methionine by a predetermined amount per day. According to this embodiment, the subject's intake of methionine is reduced by 2,000 mg, 1,400 mg, 1,000 mg, 800 mg, 500 mg, 300 mg, 200 mg, 100 mg, 80 mg, 60 mg, 50 mg, 25 mg or 10 mg or more per day.

In another aspect, the invention provides methods for simultaneously reducing blood homocysteine levels in a mammal while supplying all or part of the subject's daily mineral requirements. In this aspect, the method comprises administering a mineral chelate of HMTBA in a dose sufficient to reduce the homocysteine level of the blood and deliver at least a portion of the subject's daily requirement of methionine or an essential mineral. In one embodiment, the mineral chelate is administered in a dose sufficient to deliver at least 10% of the subject's daily requirement of an essential mineral. The essential mineral can be selected from the group consisting of calcium, copper, zinc, magnesium, manganese, iron or selenium.

In one embodiment, the method comprises administering a combination of HMTBA and other active agents to further facilitate the lowering of homocysteine levels, such as co-factors required for the metabolism of homocysteine. Such active agents include, for example, vitamin B12 and folic acid, which have been shown to affect blood homocysteine levels. Thus, in one embodiment, the method comprises administering a composition comprising HMTBA and a vitamin or co-factor selected from the group consisting of vitamin B6, folic Acid, vitamin B12, and inositol, betaine, trimethylglycin, cyanobalamin, cystine, serine, copper, and other B-group vitamins. While the optimum dose will depend on the diet, health, and age of the subject and other environmental or genetic factors, typical dosages of the vitamins and co-factors, including multivitamin and mineral supplements, are as described above.

In another embodiment, the method comprises further administering cystine and/or serine. Cystine can be administered at a dosage between 150 mg and 3,000 mg, for example between 200 mg and 2,000 mg, or between 400 mg and 1,300 mg. Likewise, serine can be administered at a dosage between 500 mg and 16,000 mg, for example between 1,000 mg and 8,000 mg, or between 2,000 mg and 4,000 mg.

It will be clear to one of skill in the art that, where a combination of HMTBA and other compounds is administered, HMTBA can be administered prior to, simultaneously, or following administration of the other compounds.

V. Methods for Detecting Homocysteine

The presence and level of homocysteine in a biological sample can be detected and measured using a wide variety of means, including techniques to quantitate total homocysteine (Hcy) and methods for distinguishing between the free (reduced and disulfide) and protein-bound (primarily albumin) forms. Such methods are described, for example, in Ueland, et al., Clin. Chem. 39(9):1764-1779 (1993), which is incorporated by reference in its entirety herein. An enzymatic assay for homocysteine is described in U.S. Pat. No. 6,063,581, where homocysteine is assayed indirectly by measuring the product concentration following the enzyme catalyzed conversion of homocysteine to S-adenosyl homocysteine. High performance liquid chromatographic (“HPLC”) methods for Hcy and Cys are also known in the art. This analytical method discriminates between Hcy and Cys by differential adsorption and elution of the compounds on a chromatographic support. Andersson, et al., (1993) Clin. Chem. 39(8):1590-1597 describes the determination of total, free and reduced Hcy and Cys. Hcy and Cys analysis by means of a gas chromatograph-mass spectrometer is described in U.S. Pat. No. 4,940,658. PCT/US92/05727 describes a chromatographic assay for cystathionine, the intermediary amino acid between Hcy and Cys produced in the metabolism of methionine. Fiskerstrand, et al., Clin. Chem. 39(2):263-271 (1993) describes a fully automated analysis of total Hcy involving fluorescent labeling of serum thiols, followed by chromatographic separation of the Hcy derivative from the other sulfur-containing compounds.

Identification of homocysteine by HPLC methods often involves derivatization with fluorescent reagents, or a radioenzymatic technique (Refsum, et al., (1985) Clin. Chem. 31(4) 624-628). In addition, identification of Cys by protein sequence analysis involves derivatization with alkylating reagents (Jue, et al., (1993) Analytical Biochemistry 210:39-44).

Techniques for handling undesirable cross-reactants are known and described in the art, for example, in U.S. Pat. No. 4,952,336, which describes a method of pre-treating a sample with an aqueous periodate solution to eliminate cross-reactants in an amphetamine-methamphetamine immunoassay. PCT/GB90/01649 discloses to an immunoassay wherein the level of interference from rheumatoid factor is reduced by pretreating the sample with a reducing agent. U.S. Pat. No. 4,978,632 discloses an immunoassay wherein the level of interference from blood and blood products is eliminated by pretreating the sample with an oxidizing agent. These pretreatment methods only affect the cross-reactants; none of the methods affect the analyte.

VI. Methods of Treatment

The present invention provides methods for treating or preventing diseases and disorders associated with elevated homocysteine levels in a subject. Such diseases and disorders include, without limitation, hyperhomocysteinemia, hyperhomocysteineuria, occlusive vascular diseases such as myocardial infarction, stroke, pulmonary embolism, occlusive disease of cerebral, carotid and aorto-iliac vessels, Alzheimer's disease, and neurological degeneration. In general, the methods of the present invention are useful for treating or preventing diseases and disorders that are currently known to be associated with elevated homocysteine levels in a subject, as well as diseases and disorders that become known or shown to be associated with elevated homocysteine levels in the future. The methods of the present invention may be used alone or in conjunction with treatment regimens for other (i.e., non-homocysteine associated) indications including, for example, treatment of proliferative diseases and disorders (e.g., cancer), diabetes, renal insufficiency, and obesity.

In another aspect, the methods of the present invention include the treatment of a disease associated with elevated homocysteine and/or reducing a subject's total methionine intake while simultaneously supplying all or part of the subject's daily mineral requirements

Treatment of Vascular Diseases

Numerous animal models for the study of vascular diseases exist. For example, U.S. Pat. No. 2003/0140357 describes a non-human transgenic mammal that exhibits an atherosclerosis phenotype as a consequence of the expression of a transgene encoding mammalian cholesteryl ester transfer protein (CETP). Additional examples of animal models for the study of atherosclerosis and other vascular diseases include U.S. Pat. App. No. 2001/0039666, U.S. Pat. App. No. 2002/0108131, and U.S. Pat. No. 6,156,727, all of which are incorporated herein by reference.

Two recent studies have been used to show a causal link between elevated homocysteine levels and development of atherosclerotic lesions in animal models (Hoffmann et al., (2001) J. Clin. Invest. 107: 675-683; Zhou et al., (2001) Arterioscler. Thromb. Vasc. Biol. 21:1470-1476). The study by Hoffmann et al. induced hyperhomocysteinemia in apoE-null mice by methionine supplementation. After three months, the mice that were fed the hyperhomocysteinemic diets had significantly larger mean aortic lesion areas compared with apoE-null mice that were fed a control diet.

The study performed by Zhou et al., which also employed transgenic mice deficient in apoE, differed from the study by Hoffmann, in that the Zhou study employed a high-fat diet, in addition to a dose of methionine or homocysteine to induce hyperhomocysteinemia. After three months, the hyperhomocysteinemic mice had significantly larger mean aortic lesion areas compared with controls.

The efficacy of HMTBA in the treatment of atherosclerosis and related indications can be determined in such an animal system. Treatment using HMTBA, either alone or in combination with other agents, can be administered over three months in transgenic apoE-deficient mice treated to induce hyperhomocysteinemia, either in a high-fat or low-fat diet. Progress or atherosclerosis can be monitored by means of several indicators, including atherosclerotic lesion size, VCAM-1 induction, and RAGE induction (Hoffmann et al., id). Additional methods, such as laser Doppler blood flow (LDBF) (Paris et al., (2003) Neurol Res. 25:642-651), angiography (Elner et al., (2005) Invest Ophthalmol Vis Sci. 46:299-303) can be employed to measure any changes in blood flow.

Alternatively, mouse models of hyperhomocysteinemia are known (Gilfix, (2003) Clin Invest Med. 26:121-32), and can be used to test the efficacy of HMTBA and its chelates in reducing homocysteine levels. For example, mice with mutations in the enzyme 5,10 methylene tetrahydrofolate reductase (MTHFR), involved in folate synthesis, can be used. Transgenic mice expressing human alleles of the MTHFR gene found in hyperhomocysteinemic patients (Blom (2000) Eur. J. Pediatr. 159 Suppl 3:S208-12) can be introduced into transgenic animal models.

Elevated intracellular homocysteine concentrations with corresponding increases in blood levels can result from increased homocysteine production or reduced metabolism. Severe hyperhomocysteinemia is rare; however, mild hyperhomocysteinemia is thought to occur in 5 to 7% of the general population. Patients with mild hyperhomocysteinemia are asymptomatic until the third or fourth decade of life when premature coronary artery disease may develop, as well as recurrent arterial and venous thrombosis. Using any of the standard analytical methods, serum homocysteine levels of between 5 and 15 μmol/L are generally considered normal in the fasting state. Hyperhomocysteinemia have been classified as moderate (15 to 30 μmol/L), intermediate (>30 to 100 μmol/L) and severe (>100 μmol/L), measured during fasting (Kang et al., Ann Rev Nutr 1992; 12: 279-298).

Deficiencies in vitamin cofactors (folate, vitamin B₁₂ and vitamin B₆) necessary for homocysteine metabolism may also promote hyperhomocysteinemia. It has been proposed that these nutritional deficiencies contribute to up to two-thirds of all cases of hyperhomocysteinemia. In addition, several drugs, including methotrexate, theophylline, cyclosporine and most anticonvulsants, as well as certain chronic disease states (liver and renal disease, hypothyroidism and malignancies) can lead to moderate hyperhomocysteinemia.

Patients with severely elevated homocysteine levels usually have very high fasting blood levels of homocysteine (up to 200 micromolar or more in homozygotes), and have reduced life expectancy due to early vascular complications. This rare condition is distinct from other milder but chronic forms of homocysteinemia, the latter of which can arise from additional causes—both external and internal. Moderate hyperhomocysteinemia is possibly of much greater clinically importance due to its prevalence.

Inadequate metabolic status individually of vitamin B6, folate and vitamin B12 have been recognized as determinants of heart and peripheral occlusive disease. At the same time, deficiencies (individually) of each of these vitamins have also been known to be associated with increased homocysteine levels. Thus vitamin B6 deficient humans have a 43% reduction in cystathionine β-synthase (CBS) activity and they excrete increased quantities of homocysteine in the urine, reflecting the effect of an inadequate B6 status on homocysteine blood levels. A negative correlation exists between dietary B6 intake and blood levels of protein bound homocysteine.

Alzheimer's Disease:

The methods described herein can also be employed for the treatment of Alzheimer's disease. Hyperhomocysteinemia has been associated with a number of diseases affecting cognition (Seshadri et al., (2002) NE J. Med 346:476-483). Moderately elevated serum homocysteine is associated with an increased risk of atherothrombotic vascular events. Accordingly, serum homocysteine is increased in patients with vascular dementia but is also increased in clinically diagnosed and histologically confirmed AD (NcCaddon et al., (2002) Neurology. 58:1395-9). Homocysteine has further been proposed to potentiate beta-amyloid neurotoxicity (Ho et al., (2001) J. Neurochem. 78:249-53). Therefore, disclosed herein is a method for treating Alzheimer's disease, comprising administering an effective amount of HMTBA in a dose sufficient to reduce homocysteine levels.

As previously described, HMTBA can be administered alone, or in combination with other agents which help reduce serum homocysteine levels. In addition, in the treatment of cognitive diseases, the method can comprise administering HMTBA in combination with agents already in use for the treatment of such diseases, such as, for example, but not limited to cholinesterase inhibitors such as tacrine, muscarinic receptor agonists, inhibitors of β-amyloid production, and/or inhibitors of neurofibrillary tangle formation.

A number of in vitro methods have been described for studying AD-related neurodegeneration. U.S. Pat. App. Ser. No. 2005/0090441, which is incorporated herein in its entirety by reference, describes the use of AD7c-NTP cDNA on post-mitotic primary rat cerebellar neuron (rCBN) cultures to accelerate neurodegeneration. Efficacy of HMTBA can be tested to see its effect in slowing or stopping such neurodegeneration.

Numerous examples of animal models of Alzheimer's disease are known in the art and are described, for example, in U.S. Pat. No. 6,717,031, which describes the selection of a transgenic mouse as a model for Alzheimer's disease by expression of the human APP bearing mutations. Other models are described, for example, in U.S. Pat. No. 5,877,399, U.S. Pat. No. 5,811,633, and U.S. Pat. No. 5,612,486, all of which are incorporated herein in their entirety.

Useful AD markers include: Glial fibrillary acidic protein (GFAP), a marker which increases in AD brain tissue; Detection of Gliosis, also associated with the neuropathology of Alzheimer's disease; Measurement of Cholinergic Nerve Terminals (a population of cholinergic neurons projecting to the forebrain have been shown to be selectively decreased in the postmortem brains of patients diagnosed with Alzheimer's disease); Measurement of Sodium-Potassium ATPase (AD is characterized by massive neurodegeneration and reduction in ouabain-binding sites, corresponding to the neuronal isoforms of this enzyme), to name a few.

Therefore, similar to the previously described method to test efficacy of HMTBA, either alone or in combination with other agents, in the treatment of vascular diseases, the animal models described herein for Alzheimer's disease can be administered with HMTBA at varying doses and compared with control animals not administered with HMTBA. Progression of the disease can be monitored using amyloid plaque deposition, other the markers described above.

Additional, less invasive behavioral and cognitive tests can also be performed, for example, as described in U.S. Pat. App. Ser. No. 2003/0101467, which is incorporated in its entirety. Transgenic mice models of AD often show significant cognitive and/or behavioral changes, corresponding to changes observed with patients suffering from AD (Mega et al. (1996) Neurology 46, 130-135). For example, in the Half-Enclosed Platform test (Kasermann (1986) Psychopharmacol. 89, 31-37), water maze test (Morris et al. (1982) Nature 297, 681-683).

VII. Methods of Nutrition

The present invention is based partly on the surprising discovery that metal complex compositions containing 2-hydroxy-4-(thiomethyl)-butanoic acid can serve as effective sources of both mineral and methionine in humans. Although not wishing to be bound by any theory, minerals within certain mineral complexes containing 2-hydroxy-4-(thiomethyl)-butanoic acid are tightly coordinated, and in some cases covalent, complexes which show ideal dissociation properties, allowing for enhanced absorption into the body when compared with inorganic salts of minerals. Therefore, in another aspect, the invention provides a method of administering a mineral to a human, comprising administering a mineral chelate composition comprising 2-hydroxy-4-(thiomethyl)-butanoic acid. The mineral is preferably an essential mineral, and can be selected from the group consisting of calcium, magnesium, zinc, selenium, manganese, iron, and copper. In one embodiment, the mineral chelate composition is calcium-HMTBA. In another embodiment, the mineral chelate composition is zinc-HMTBA.

In still another aspect, the invention provides a method of preventing and/or treating a condition associated with mineral deficiency in a human or other animal subject comprising orally administering an effective amount of a mineral comprising a salt or complex selected from the group consisting of calcium-2-hydroxy-4-(thiomethyl)-butanoic acid, zinc-2-hydroxy-4-(thiomethyl)-butanoic acid, magnesium-2-hydroxy-4-(thiomethyl)-butanoic acid, manganese-2-hydroxy-4-(thiomethyl)-butanoic acid, selenium-2-hydroxy-4-(thiomethyl)-butanoic acid, copper-2-hydroxy-4-(thiomethyl)-butanoic acid, and iron-2-hydroxy-4-(thiomethyl)-butanoic acid. In order to prevent and/or treat a condition associated with deficiency of more than one mineral, the method can comprise administering an effective more than one salt or complex. In one embodiment, the method comprises administering at least 50% of the U.S. recommended daily allowance (RDA) of the mineral, for example, at least 50%, 60%, 75%, 100% or more (Food and Nutrition Board/National Research Council. 1989. Recommended Dietary Allowances. 10th ed. Washington, National Academy Press). In one embodiment, the method comprises administering a safe and effective amount of a mineral.

VIII. Pharmaceutical Compositions

The pharmaceutical preparations disclosed herein are prepared in accordance with standard procedures and are administered at dosages that are selected to reduce, prevent, or eliminate cancer, or to provide a protective effect against genotoxic agents such as ionizing radiation. (See, e.g., Remington's Pharmaceutical Sciences, Mack Publishing Company, Easton, Pa.; and Goodman and Gilman, Pharmaceutical Basis of Therapeutics, Pergamon Press, New York, N.Y., the contents of which are incorporated herein by reference, for a general description of the methods for administering various antimicrobial agents for human therapy). The compositions of the present invention can be delivered using controlled (e.g., capsules) or sustained release delivery systems (e.g., biodegradable matrices). Examples of delayed release delivery systems for drug delivery suitable for administering compositions of the invention are described in U.S. Pat. Nos. 4,452,775, U.S. Pat. No. 5,239,660, and U.S. Pat. No. 3,854,480.

The pharmaceutically acceptable compositions of the present invention comprise one or more compounds of the present invention in association with one or more non-toxic, pharmaceutically acceptable carriers and/or diluents and/or adjuvants and/or excipients, collectively referred to herein as “carrier” materials, and if desired other active ingredients. The compositions may contain common carriers and excipients, such as corn starch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Tablet binders that can be included are acacia, methylcellulose, sodium carboxymethylcellulose, polyvinylpyrrolidone (Providone), hydroxypropyl methylcellulose, sucrose, starch and ethylcellulose.

Lubricants that can be used include magnesium stearate or other metallic stearates, stearic acid, silicon fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product comprising a compound of the present invention.

For oral use, solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica or talc: disintegrants, for example, potato starch, flavoring or coloring agents, or acceptable wetting agents. Oral liquid preparations generally are in the form of aqueous or oily solutions, suspensions, emulsions, syrups or elixirs and may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The amount of the compound of the present invention in a unit dosage comprises a therapeutically-effective amount of at least one active compound of the present invention which may vary depending on the recipient subject, route and frequency of administration. A subject refers to an animal such as an ovine or a mammal, including a human.

According to this aspect of the present invention, the novel compositions disclosed herein are placed in a pharmaceutically acceptable carrier and are delivered to a recipient subject (including a human subject) in accordance with known methods of drug delivery. In general, the methods of the invention for delivering the compositions of the invention in vivo utilize art-recognized protocols for delivering the agent with the only substantial procedural modification being the substitution of the compounds of the present invention for the drugs in the art-recognized protocols.

The compounds of the present invention can be administered as a single daily dose or in multiple doses per day. The treatment regime may require administration over extended periods of time, e.g., for several days or for from two to four weeks. The amount per administered dose or the total amount administered will depend on such factors as the nature and severity of the disease condition, and the age and general health of the recipient subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Zinc-HMTBA Fortified Energy Bars

The following formulations for three different energy bars show products with Zinc-HMTBA. Mineral chelates of HMTBA can be prepared, for example, as described in U.S. patent application Ser. No. 11/074,387, U.S. Pat. No. 2,745,745, U.S. Pat. No. 2,938,053, U.S. Pat. No. 4,579,962, U.S. Pat. No. 4,335,257, U.S. Pat. No. 6,461,664,), U.S. Pat. No. 5,583,243 and U.S. Ser. No. 11/074,387, the contents of which are incorporated herein in their entirety. The mineral chelates of HMTBA thus prepared can be used directly, or further purified by chromatography, re-crystallization, organic solvent extraction, or by adsorption of contaminants using activated carbon.

Ingredients for Milk Chocolate Peanut Butter Bar

8% Zinc-HMTBA chelate

13% soy protein isolate

8% whey powder

5% 10 D.E. maltodextrin

12% crystalline fructose

10% sucrose

2% nonfat dry milk

13% corn syrup 42 D.E.

2% peanut flour

6% peanut butter

4% partially hydrogenated soybean oil

2% honey

5% densified crisp rice #110

0.1% salt

0.5% lecithin

0.6% vitamin & mineral blend

0.4% butter vanilla flavor

0.4% natural flavor blend

8% water

Ingredients for Black & White Chocolate Bar

8% Zinc-HMTBA chelate

13% soy protein isolate

8% whey powder

8% 10 D.E. maltodextrin

13% crystalline fructose

10% sucrose

3% nonfat dry milk

13% corn syrup 42 D.E.

5% dark cocoa

4% partially hydrogenated soybean oil

2% honey

5% densified crisp rice

0.1% salt

0.5% lecithin

0.6% vitamin & mineral blend

0.4% butter vanilla flavor

0.4% natural flavor blend

6% water

Ingredients for DBL Dark Chocolate Crunch Bar

8% Zinc-HMTBA chelate

13% soy protein isolate

8% whey powder

6% 10 D.E. maltodextrin

15% crystalline fructose

10% sucrose

3% nonfat dry milk

13% corn syrup 42 D.E.

5% dark cocoa

4% partially hydrogenated soybean oil

2% honey

5% densified crisp rice

0.1% salt

0.5% lecithin

0.6% vitamin & mineral blend

0.4% butter vanilla flavor

0.4% natural flavor blend

6% water

The general procedure for preparing these energy bars is as follows: First, in a blend tank, a slurry of water, corn syrup, sucrose, fructose, soybean oil and honey is formed. To this slurry, either peanut butter (milk chocolate peanut butter bar) or dark cocoa (black and white chocolate bar or DBL dark chocolate bar) is added. The slurry is then heated up to 120° F. and placed in a dough mixer. Other dry ingredients are then added to the slurry and the batch is mixed until homogenous. Next, flavors and crisp rice are added and mixed until dispersed.

The resulting mass is then loaded into an extruder and extruded to a predetermined size. The extruded bars are then run under refrigerated air blast to cool. Once cooled, the bars are coated with milk chocolate (milk chocolate peanut butter bar), white chocolate (black and white chocolate bar) or dark chocolate containing crisp rice (DBL dark chocolate crunch bar). The weight ratio of chocolate coating to extruded center is 1:2 (or 50 pounds of chocolate coating to 100 pounds of extruded center).

### Example 2

### Calcium HMTBA Fortified Energy Bar

Ingredients for Black & White Chocolate Bar

12% Ca HMTBA chelate

13% soy protein isolate

8% whey powder

6% 10 D.E. maltodextrin

11% crystalline fructose

10% sucrose

3% nonfat dry milk

13% corn syrup 42 D.E.

5% dark cocoa

4% partially hydrogenated soybean oil

2% honey

5% densified crisp rice

0.1% salt

0.5% lecithin

0.6% vitamin & mineral blend

0.4% vanilla flavor

0.4% natural flavor blend

6% water

The procedure for preparing the black and white energy bar is as follows: First, in a blend tank, a slurry of water, corn syrup, sucrose, fructose, soybean oil and honey is formed. The slurry is heated up to 120° F. and placed in a dough mixer where the other ingredients are added and mixed until homogenous. Next, flavors and crisp rice are added and mixed until dispersed. The resulting mass is then loaded into an extruder and extruded to a predetermined size. The extruded bars are then run under refrigerated air blast to cool. Once cooled, the bars are coated with white chocolate. The weight ratio of chocolate coating to extruded center is 1:2 (or 50 pounds of chocolate coating to 100 pounds of extruded center). Once tempered, the finished bar may be packaged.

While the invention has been described with reference to certain preferred embodiments, those skilled in the art will appreciate that various modifications, changes, omissions, and substitutions can be made without departing from the spirit of the invention. For example, the HMTBA chelates of the present invention may be used to fortify other foods and/or drinks such as weight loss bars, chewable tablets, etc. Further, HMTBA chelates having other chelated metals than those described above may be prepared by following similar procedures as would be apparent to those skilled in the art.

1. A method of treating a disease or disorder associated with elevated homocysteine levels in a subject, comprising administering HMTBA, or a salt, chelate, or complex thereof, in a dose sufficient to lower said homocysteine levels.

2. The method of claim 1, wherein the disease or disorder is selected from the group consisting of cardiac disease, ischemia, and Alzheimer's disease.

3. The method of claim 1, wherein the subject is not being treated for renal insufficiency.

4. The method of claim 1, wherein a metal complex of HMTBA is administered.

5. The method of claim 4, wherein a zinc complex of HMTBA is administered.

6. The method of claim 1, wherein the administered HMTBA, or salt, chelate, or complex thereof, accounts for at least 20% of the subject's total methionine intake.

7. The method of claim 1, further comprising administering to said subject an effective amount of at least one compound selected from the group consisting of vitamin B6, folic acid, vitamin B12, zinc, N-acetyl cysteine, and inositol.

8. The method of claim 1, wherein a mineral chelate of HMTBA is administered in a dose sufficient to lower said homocysteine levels and provide at least 10% of the subject's daily requirement of methionine or an essential mineral.

9. The method of claim 8, wherein said mineral chelate of HMTBA accounts for at least 20% of the subject's total methionine intake.

10. A method of administering a mineral to a human, comprising administering a composition comprising a mineral chelate of 2-hydroxy-4-(thiomethyl)-butanoic acid.

11. The method of claim 10, wherein said mineral chelate comprises a mineral selected from the group consisting of calcium, magnesium, manganese, copper, iron, selenium and zinc.

12. A method of preventing or treating a condition associated with mineral deficiency in a subject, comprising orally administering an effective amount of a mineral supplement comprising a salt or complex selected from the group consisting of calcium-2-hydroxy-4-(thiomethyl)-butanoic acid, zinc-2-hydroxy-4-(thiomethyl)-butanoic acid, magnesium-2-hydroxy-4-(thiomethyl)-butanoic acid, selenium-2-hydroxy-4-(thiomethyl)-butanoic acid, and iron-2-hydroxy-4-(thiomethyl)-butanoic acid.

13. A nutritional composition comprising a mineral chelate of HMBTA, wherein said composition is packaged in units, each of which comprises at least 50% of the U.S. recommended daily allowance (RDA) of the mineral for humans.

14. The composition of claim 13, wherein the mineral chelate is selected from the group consisting of calcium-2-hydroxy-4-(thiomethyl)-butanoic acid, zinc-2-hydroxy-4-(thiomethyl)-butanoic acid, magnesium-2-hydroxy-4-(thiomethyl)-butanoic acid, selenium-2-hydroxy-4-(thiomethyl)-butanoic acid, and iron-2-hydroxy-4-(thiomethyl)-butanoic acid.

15. The composition of claim 13, which is in the form of a chewable tablet, a non-chewable tablet, an energy bar, a sports bar, a weight loss bar, a snack bar, a granola bar, an isotonic beverage, a drink mix, an energy drink, a sports drink, a citrus drink, a fruit drink, or a carbonated drink.

16. The energy bar of claim 15 comprising about 8% zinc-2-hydroxy-4-(thiomethyl)-butanoic acid.

17. The energy bar of claim 15 comprising about 12% calcium-2-hydroxy-4-(thiomethyl)-butanoic acid.
